(19) United States
(12) Patent Application Publication
(10) Pub. No.: US 2015/0322489 A1
(43) Pub. Date: Nov. 12, 2015

(54) SILICA-BASED BIOLOGICAL MATERIAL ISOLATION

(71) Applicant: DOUGLAS SCIENTIFIC, LLC, Alexandria, MN (US)

(72) Inventor: Hans A. Mische, Grey Eagle, MN (US)

(73) Assignee: DOUGLAS SCIENTIFIC, LLC, Alexandria, MN (US)

(21) Appl. No.: 14/762,325

(22) PCT Filed: Jan. 23, 2014
(86) PCT No.: PCT/US14/12726

§ 371 Date: Jul. 21, 2015

Related U.S. Application Data
(60) Provisional application No. 61/756,703, filed on Jan. 25, 2013.

(51) Int. Cl. C12Q 1/68; G01N 1/40

(52) U.S. Cl. CPC C12Q 1/6806; G01N 1/405

(57) ABSTRACT

An apparatus for isolating nucleic acids includes an elongated body. The elongated body includes a silica surface positioned and configured to bind the nucleic acids when the elongated body is dipped into a biological material sample.

### BACKGROUND

The present invention relates to biological material isolation, and specifically to the use of a silica-based elongated body to isolate, purify, and transfer nucleic acids.

There are a number of existing biological material purification methods. One of the most common methods is the use of silica beads and silica resins to bind and thus isolate nucleic acid molecules. The isolated nucleic acids can subsequently be amplified and analyzed via processes such as polymerase chain reaction (PCR). Chromatographic purification of nucleic acids is another common method, including silica based membrane purification, size exclusion chromatography, reversed phase chromatography, gel filtration, magnetic bead based purification, and ion-exchange chromatography. Ion-exchange chromatography is one of the most commonly used separation and purification methods for plasmid DNA, genomic DNA and RNA. A wide range of nucleic acids can also be isolated using cellulose and cellulose filter paper. However, currently available methods of biological material purification are expensive, time-consuming, and often complex and difficult to use.

### SUMMARY

An apparatus for isolating nucleic acids includes an elongated body. The elongated body includes a silica surface positioned and configured to bind the nucleic acids when the elongated body is dipped into a biological material sample.

A method of isolating nucleic acids includes dipping an elongated body including a silica surface into a biological material sample to bind the nucleic acids

### BRIEF DESCRIPTION OF THE DRAWINGS

FIG. 1 is a perspective view of two embodiments of the silica-based elongated body of the present invention, specifically a silica rod and a silica tube.

FIG. 2 is a perspective view of silica rods of the present invention in an array format.

FIG. 3 is a frontal view of a silica tube of the present invention placed in a sample well.

FIGS. 4A-4C are frontal views of different embodiments of the silica-based rod of the present invention.

FIG. 5 is an isometric view of one embodiment of a silica rod of the present invention.

FIG. 6 is an isometric view of another embodiment of a silica rod of the present invention.

FIG. 7 is a frontal view of the silica-based elongated body apparatus of the present invention with reaction and detection capabilities.

FIG. 8 is a frontal view of the silica-based elongated body apparatus of the present invention with concave and convex lenses as well as reaction and detection capabilities.

FIG. 9 is a frontal view of the silica-based elongated body apparatus of the present invention with magnetic capabilities.

FIG. 10 is a frontal view of the silica-based elongated body apparatus of the present invention with additional functionalized regions.

FIG. 11 is a frontal view of the silica-based elongated body apparatus of the present invention with electrodes for attracting molecules in addition to nucleic acids.

FIG. 12 is a frontal view of another embodiment of the silica-based elongated body apparatus of the present invention with electrodes for attracting molecules in addition to nucleic acids.

### DETAILED DESCRIPTION

The nucleic acid purification apparatus of the present invention includes a silica-based elongated body for binding, purifying, and transferring nucleic acids. The silica-based elongated body may be a solid rod or hollow tube and may be additionally functionalized for targets other than nucleic acids. The apparatus may be used singularly or in arrays. The silica-based elongated body of the present invention may be integrated into an automated system for high throughput processing.

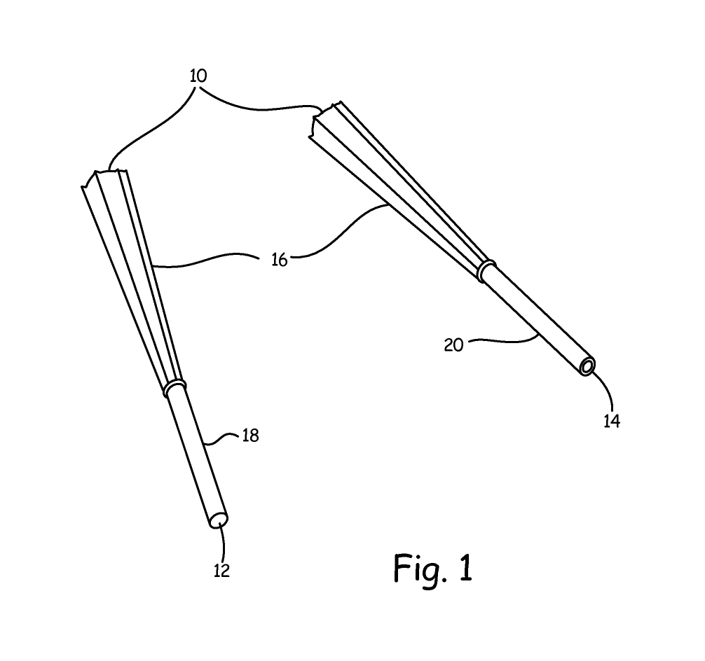

FIG. 1 a perspective view of modified pipette tips 10, including silica solid rod 12 and silica hollow tube 14. Silica solid rod 12 includes pipette tip 16 and silica solid cylinder 18. Silica hollow tube 14 includes pipette tip 16 and silica hollow cylinder 18. In one embodiment, silica solid rod 12 may be dipped into a biological material sample to bind desired biological material to the surface of silica solid cylinder 18. In an alternative embodiment, silica hollow tube 14 may be dipped into a biological material sample to bind desired biological material to inside and outside surfaces of silica hollow cylinder 20.

In another embodiment, silica hollow tube 14 may be used to perform one or more aspirations to increase amount of nucleic acid bound to silica hollow tube 14. In another embodiment, silica hollow tube 14 may be connected to a pipettor to aid in manipulation of sample fluid and purification automation. In alternative embodiments, the silica-based elongated body of the present invention may be any suitable solid or hollow shape with a triangular, square, or other polygonal cross section. The surface of the silica-based elongated body may be convoluted, undulating, or any other suitable shape that increases the surface area of the silica-based elongated body.

To form the silica-based elongated body of the present invention, silica or other suitable silica-based materials may be adhered, thermally or mechanically, or may be embedded into surfaces of a solid rod or hollow tube. In alternative embodiments, the silica-based elongated body may be molded or extruded from silica into the desired tube or rod shape. In further alternative embodiments the elongated body of the present invention may include other suitable materials such as metal tubing, mandrels, wires, ceramics, carbon, or polymers. In other embodiments, the surface of the silica may be chemically etched with a chemical such as hydrofluoric acid (HF) or could be plasma etched in order to increase the nucleic acid affinity of the silica-based elongated body.

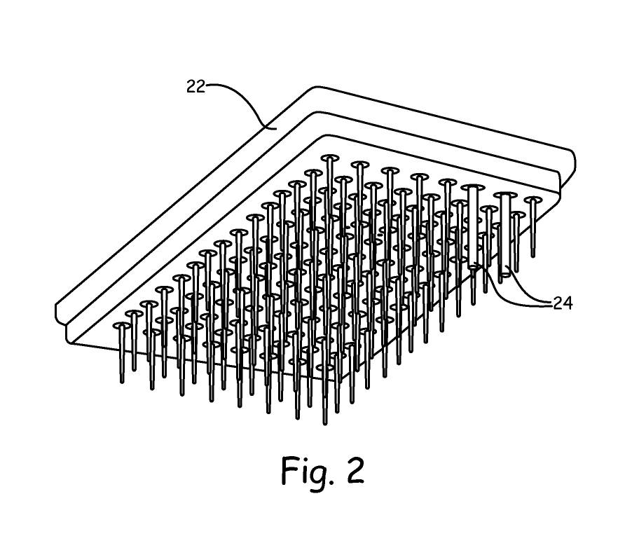

FIG. 2 is a perspective view of array 22 with 96 silica solid rods 24. In one embodiment of the present invention, biological material samples are placed in a 96 well array. Silica solid rods 24 are dipped into the samples to bind nucleic acids to silica solid rods 24. Silica solid rods 24 may then be agitated in order to facilitate nucleic acid harvesting. Silica solid rods 24 may be subsequently washed to remove any contaminants and leave only the nucleic acids bound to silica solid rods 24. Silica solid rods 24 may be agitated during the wash process in order to facilitate contaminant removal.

Silica solid rods 24 are then dipped into an elution buffer to elute the nucleic acids from silica solid rods 24. Silica solid rods 24 may be agitated during the elution process in order to facilitate elution. In alternative embodiments, array 22 may include 384 silica solid rods 24, 1536 silica solid rods 24, or any other suitable number of silica solid rods 24. In another alternative embodiment, silica solid rods 24 may be replaced by silica hollow tubes, and once the desired biological material is eluted, the desired biological material may also be aspirated with the silica hollow tubes for transfer to a processing module.

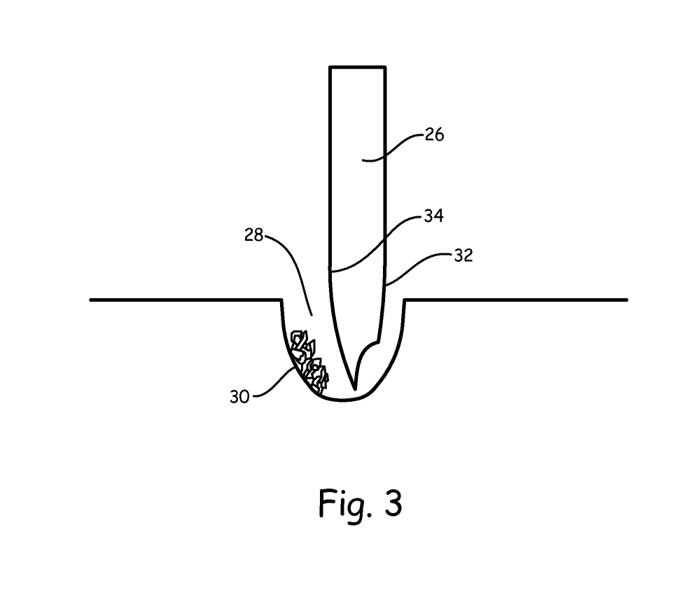

FIG. 3 is a frontal view of silica hollow tube 26 dipped into sample well 28 with biological material sample 30. Silica hollow tube 26 includes outer surface 32 and inner surface 34, which increase the over-all surface area of silica hollow tube 26 available for binding nucleic acids. Silica tube 26 may also be used as a pipette tip for fluid handling and transfer. In one embodiment, silica hollow tube 26 is dipped into sample 28 to bind nucleic acids from biological material sample 30. The nucleic acids from biological material sample 30 bind to both outer surface 32 and inner surface 34 of silica hollow tube 26. Silica hollow tube 26 may subsequently be dipped in an elution buffer to elute the nucleic acids. Silica hollow tube 26 may then be used as a pipette to aspirate the eluted nucleic acids and transfer the nucleic acids to a detection station.

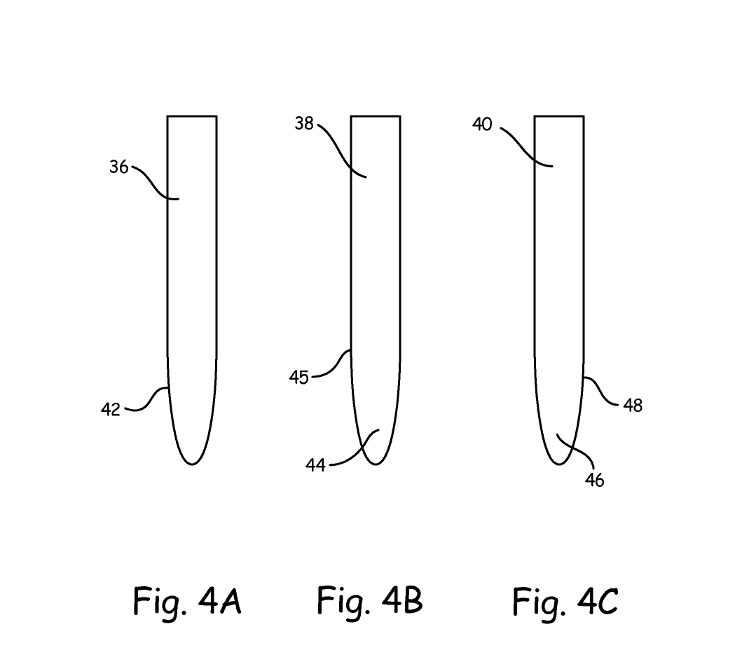

FIGS. 4A-4C are frontal views of three embodiments of the silica-based elongated body of the present invention, including silica solid rod 36, silica hollow tube 38, and silica hollow tube 40, respectively. Referring to FIG. 4A, silica solid rod 36 includes outer surface 42. Silica solid rod 36 may be formed by coating outer surface 42 with silica. Silica solid rod 36 may bind nucleic acids to outer surface 42.

Referring to FIG. 4B, silica hollow tube 38 includes inner surface 44 and outer surface 45. Silica hollow tube 38 may be formed by coating inner surface 44 with silica. Silica hollow tube 38 includes silica on inner surface 44 and not on outer surface 45; therefore silica hollow tube 38 binds nucleic acids only to inner surface 44. In an alternative embodiment, silica hollow tube 38 may include silica on outer surface 45 and not on inner surface 44; therefore silica hollow tube may bind nucleic acids only to outer surface 45. Since silica hollow tube 38 is hollow, silica hollow tube 38 may also be used as a pipette for aspirating and dispensing nucleic acids after they have been isolated.

Referring to FIG. 4C, silica hollow tube 40 includes inner surface 46 and outer surface 48. Silica hollow tube 40 may be formed by coating inner surface 46 and outer surface 48 with silica. Silica hollow tube 40 includes silica on inner surface 46 and outer surface 48; therefore silica hollow tube 40 binds nucleic acids to both inner surface 46 and outer surface 48. Silica hollow tube 40 may also be used as a pipette for aspirating and dispensing nucleic acids after they have been isolated.

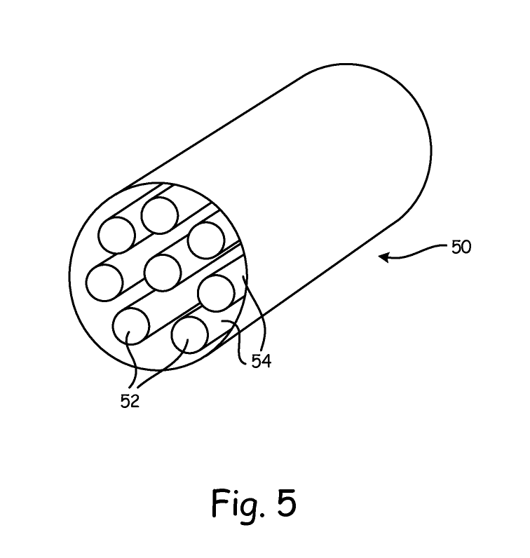

FIG. 5 is an isometric view of tube 50. Tube 50 includes interlumenal silica solid rods 52 with outer surfaces 54. Interlumenal silica solid rods 52 may bind nucleic acids to outer surfaces 54. In alternative embodiments, interlumenal silica solid rods 52 may be silica fibers or another suitable shape. When tube 50 is dipped into a biological material sample, outer surfaces 54 of interlumenal silica solid rods 52 will bind nucleic acids to isolate the nucleic acids. Interlumenal silica solid rods 52 provide an increased surface area for binding nucleic acids and thus may increase the nucleic acid purification efficiency of tube 50.

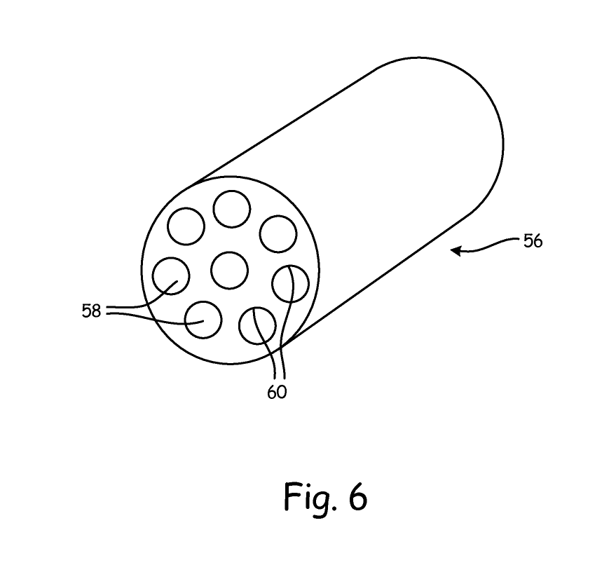

FIG. 6 is an isometric view of silica rod 56. Silica rod 56 includes lumens 58 with inner surfaces 60. Inner surfaces 60 of lumens 58 may bind nucleic acids. When silica rod 56 is dipped into a biological material sample, inner surfaces 60 of lumens 58 will bind nucleic acids to isolate the nucleic acids. Lumens 58 provide an increased surface area for binding nucleic acids and thus may increase the nucleic acid purification efficiency of silica rod 56. Additionally, once isolated, nucleic acids bound to inner surfaces 60 may be eluted. Subsequently, lumens 58 may be used to independently or simultaneously aspirate or dispense the eluted nucleic acids. Lumens 58 may also be used to aspirate or dispense other chemicals or reagents based on the desired chemical reaction.

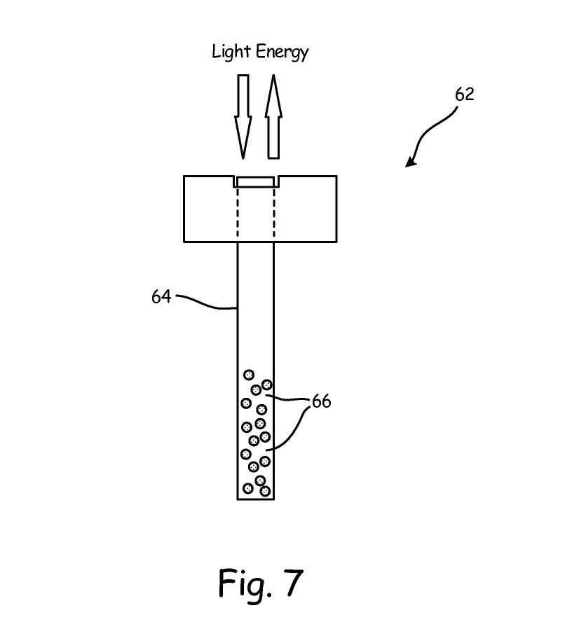

FIG. 7 is a frontal view of silica-based apparatus 62 with reaction and detection capabilities. Silica-based apparatus 62 includes silica rod 64 and bound deoxyribonucleic acid (DNA) 66. After isolating bound DNA 66 and dipping silica rod 64 in a reagent, silica rod 64 may be used as a light transmitting element in order to provide thermal energy to bound DNA 66 and the reagent to perform and complete a chemical reaction such as polymerase chain reaction (PCR), isothermal amplification, real time PCR (qPCR), loop-mediated isothermal amplification (LAMP), genetic sequencing, or any other suitable reaction. Reagents may include fluorescent, phosphorescent, chemoluminescent, or any other suitable detectable material.

In alternative embodiments, silica rod 64 may be placed in a thermal chamber or affected by an external heat source in order to facilitate a reaction on the surface of silica rod 64. In further embodiments, silica rod 64 may include an internal resistive heating element or any suitable alternative for heating silica rod 64 to carry out the desired reaction. Silica rod 64 may transmit infrared, ultraviolet, or other suitable wavelengths in order to provide thermal energy for a desired chemical process.

Following completion of the desired reaction, silica rod 64 may be energized with light at a specific wavelength, such as 488 nanometers. At this wavelength, silica rod 64 thus may be used to transmit fluorescence for detection of fluorescein amidite (FAM), VIC®, and 6-Carboxyl-X-Rhodamine (ROX), or any other suitable dyes or fluorescent compounds. The transmitted fluorescence is subsequently used for analysis of the completed reaction. In alternative embodiments, at suitable wavelengths, silica rod 64 may be used to transmit phosphorescent, chemoluminescent, or any other suitable detectable material for analysis of the completed reaction.

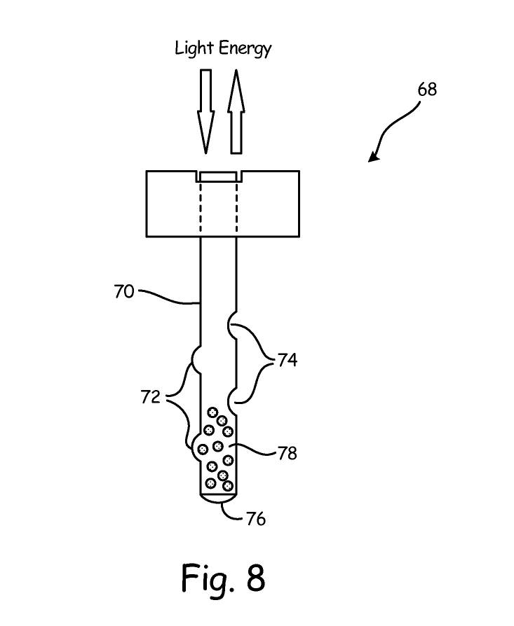

FIG. 8 is a frontal view of silica-based apparatus 68 with reaction and detection capabilities. Silica-based apparatus 68 includes silica rod 70 with convex lenses 72, concave lenses 74, tip 76, and bound nucleic acids 78. Silica rod 70 may be used to isolate bound nucleic acids 78 from a biological material sample. After bound nucleic acids 66 are isolated and bound to silica rod 70, silica rod 70 may be dipped into a reagent that binds with bound nucleic acids 78. Silica rod 70 may be used as a light transmitting element, transmitting infrared, ultraviolet, or other suitable wavelengths, in order to provide thermal energy to bound nucleic acids 78 and the reagent to perform a desired chemical process. Convex lenses 72, concave lenses 74, and tip 76 of silica rod 74 use the light transmitting capabilities of silica rod 70 to provide enhanced thermal transfer to bound nucleic acids 78 and the reagent. Tip 76 may be concave or convex. Following completion of the desired reaction, silica rod 70 may be used to transmit fluorescence, phosphorescence, or chemoluminescence for detection and analysis of a desired biological material. Silica rod 70 may magnify the emissions for enhanced detection.

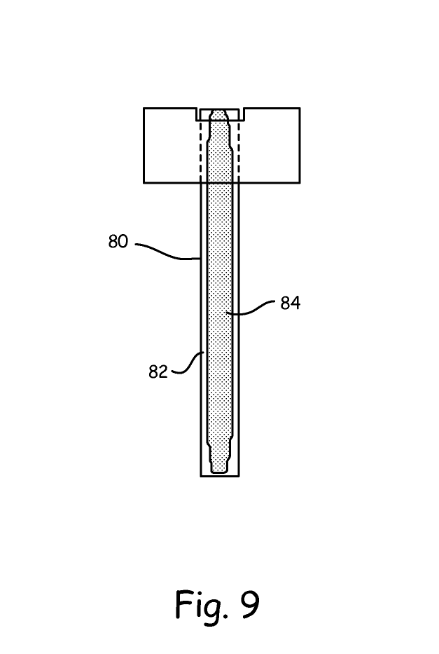

FIG. 9 is a frontal view of silica probe 80. Silica probe 80 includes silica-based rod 82 and magnetic inner layer 84. Magnetic inner layer 84 may be a permanent magnet or an electromagnet. Magnetic inner layer 84 allows for manipulation of nucleic acids and other molecular products or debris, as well as increased nucleic acid purity. In order to utilize the properties of magnetic inner layer 84, magnetic beads may be added to a biological material sample in order to bind other molecular components and debris. Other molecular products or debris, such as streptavidin, selectively bind based on their magnetic affinity. When silica probe 80 is dipped into the biological material sample, silica rod 82 binds nucleic acids. Simultaneously, magnetic inner layer 84 may cause the magnetic beads with other molecular products or debris from the biological material sample to bind to silica probe 80.

After nucleic acids and magnetic beads with other molecular products or debris are bound to silica probe 80, the nucleic acids may be eluted from silica probe 80. The magnetic beads with other molecular products or debris will remain bound to silica probe 80 due to the magnetic properties of magnetic inner layer 84. In one embodiment where magnetic inner layer 84 is a permanent magnet, the magnetic beads with other molecular products or debris may subsequently be unbound from silica probe 80 by removing magnetic inner layer 84 from silica probe 80. In another embodiment where magnetic inner layer 84 is an electromagnet, the magnetic beads with other molecular products or debris may subsequently be unbound from silica probe 80 by turning off magnetic inner layer 84.

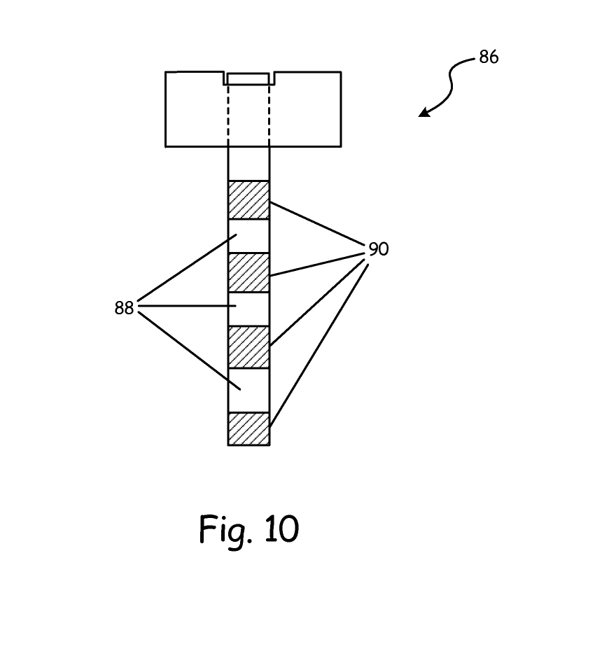

FIG. 10 is a frontal view of silica probe 86. Silica probe 86 includes bare silica regions 88 and functionalized regions 90. Silica probe 86 allows for manipulation of nucleic acids and other molecular products or debris, as well as increased nucleic acid purity. When silica probe 86 is dipped into a biological material sample, bare silica regions 88 bind nucleic acids. Functionalized regions 90 may be used to isolate other molecular components, such as proteins. Each of functionalized regions 90 may bind different molecular components based on properties such as pH. Subsequently, the bound nucleic acids may be eluted, while the bound other molecular components will remain bound to silica probe 86. The other molecular components may then be removed from functionalized regions 90.

In an alternative embodiment, silica probe 86 may be a silica tube with functionalized regions on the inner surface, outer surface, or both the inner and outer surfaces. In other embodiments, nucleotides or oligonucleotides may be deposited on silica probe 86 in a predetermined manner prior to dipping silica probe 86 into a sample. The deposited nucleotides or oligonucleotides allow for the creation or isolation of a specific gene sequence and may also be used to facilitate gene sequencing.

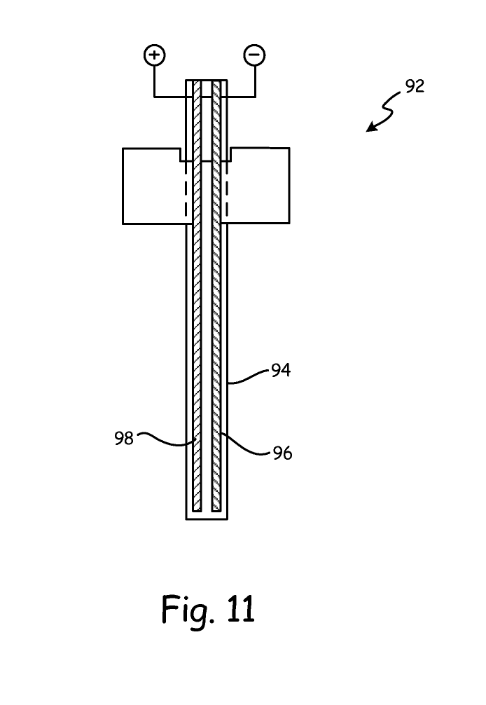

FIG. 11 is a frontal view of silica probe 92. Silica probe 92 includes silica-based rod 94, negative electrode 96, and positive electrode 98. Negative electrode 96 and positive electrode 98 may be inside of silica probe 92 or affixed to the outside of silica-based rod 94 and may be made of conductive polymers or conductors. Silica probe 92 allows for strong attraction of nucleic acids as well as attraction of other biological materials to silica probe 92. When silica probe 92 is dipped into a biological material sample, silica-based rod 94 and positive electrode 98 will bind nucleic acids. Negative electrode 96 allows silica probe 92 to bind other biological materials that have a positive charge. In alternative embodiments, the polarities of negative electrode 96 and positive electrode 98 may be reversed as desired in order to attract or repel desired biological material.

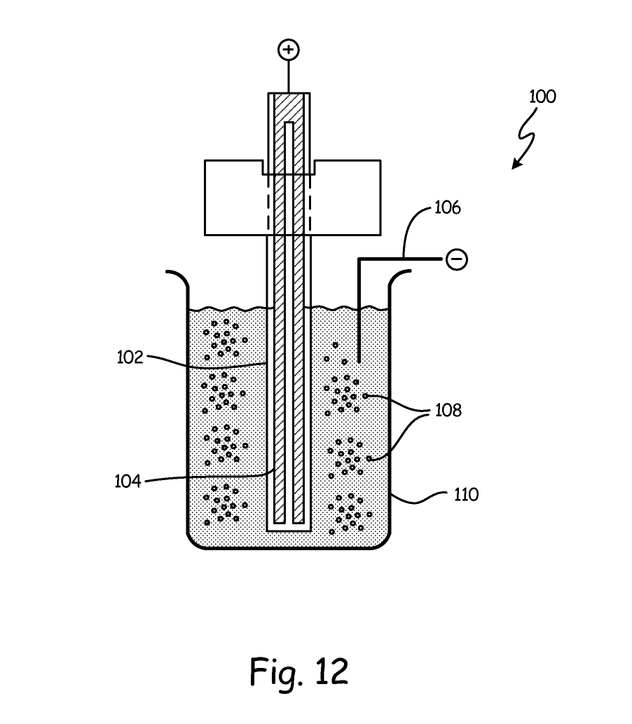

FIG. 12 is a frontal view of silica probe apparatus 100. Silica probe apparatus 100 includes silica-based rod 102, positive electrode 104, negative electrodes 106, and biological material sample 108 in beaker 110. Positive electrode 104 may be embedded in silica-based rod 102 or affixed to the outside surface of silica-based rod 102. When silica-based rod 102 and negative electrode 106 are placed in beaker 110 with biological material sample 108, negative electrode 106 repels nucleic acids and attracts positively charged biological materials from biological material sample 108. Silica-based rod 102 and positive electrode 104 attract and bind nucleic acids, isolating them from biological material sample 108. In alternative embodiments, the polarities of negative electrode 96 and positive electrode 98 may be reversed as desired in order to attract or repel desired biological material.

While the above-identified drawing figures set forth one or more embodiments of the invention, other embodiments are also contemplated. In all cases, this disclosure presents the invention by way of representation and not limitation. It should be understood that numerous other modifications and embodiments can be devised by those skilled in the art, which fall within the scope and spirit of the principles of the invention. The figures may not be drawn to scale, and applications and embodiments of the present invention may include features and components not specifically shown in the drawings.

Any relative terms or terms of degree used herein, such as “substantially”, “essentially”, “generally” and the like, should be interpreted in accordance with and subject to any applicable definitions or limits expressly stated herein. In all instances, any relative terms or terms of degree used herein should be interpreted to broadly encompass any relevant disclosed embodiments as well as such ranges or variations as would be understood by a person of ordinary skill in the art in view of the entirety of the present disclosure, such as to encompass ordinary manufacturing tolerance variations, incidental alignment variations, alignment or shape variations induced by operational conditions, and the like.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the spirit and scope of the present disclosure, viewed in its entirety.

1.-31. (canceled)

32. An apparatus comprising:
a silica-based elongated body having at least one surface that binds nucleic acids.

33. The apparatus of claim 32, wherein the elongated body has a circular cross-section.

34. The apparatus of claim 32, wherein the elongated body comprises an exterior surface and an interior surface.

35. The apparatus of claim 32, and further comprising a pipettor attached to the elongated body.

36. The apparatus of claim 32, the apparatus including a support with a plurality of silica-based elongated bodies extending from the support, each of the elongated bodies having at least one surface that binds the nucleic acids.

37. The apparatus of claim 32, wherein the elongated body includes a hollow interior with a plurality of silica-based interlumenal rods, each of the interlumenal rods having a surface that binds the nucleic acids.

38. The apparatus of claim 32, wherein the elongated body includes a plurality of lumens, each of the lumens including a silica-based inner surface that binds the nucleic acids.

39. The apparatus of claim 32, wherein the elongated body includes a plurality of convex lenses and a plurality of concave lenses.

40. The apparatus of claim 32, wherein the elongated body includes a magnetic inner layer.

41. The apparatus of claim 40, wherein the magnetic inner layer comprises an electromagnet or a permanent magnet.

42. The apparatus of claim 32, wherein the elongated body further includes a plurality of functionalized regions that bind molecular components other than nucleic acids.

43. The apparatus of claim 32, wherein the elongated body includes a negative electrode that binds biological material with a positive charge and a positive electrode that binds the nucleic acids.

44. A method of isolating nucleic acids comprising:
dipping a silica-based elongated body into a biological material sample to bind the nucleic acids.

45. The method of claim 44, and further comprising dipping the elongated body into an elution buffer to release the nucleic acids from the elongated body.

46. The method of claim 45, and further comprising aspirating the nucleic acids with the elongated body.

47. The method of claim 46, and further comprising dispensing the nucleic acids with the elongated body.

48. The method of claim 44, and further comprising dipping the elongated body into a reagent.

49. The method of claim 48, and further comprising providing thermal energy with the elongated body to the bound nucleic acids and the reagent to facilitate a chemical reaction.

50. The method of claim 49, and further comprising energizing the elongated body with light.

51. The method of claim 50, and further comprising detecting fluorescence, chemoluminescence, or phosphorescence of the chemical reaction.
